(19) United States
(12) Patent Application Publication
(10) Pub. No.: US 2025/0144017 A1
(43) Pub. Date: May 8, 2025

(54) COMPOSITION OF ORAL SOLUBLE FILM AND APPLICATION THEREOF

(71) Applicant: SHENZHEN RELX TECHNOLOGY CO., LTD., ShenZhen (CN)

(72) Inventors: YUANYUAN WANG, ShenZhen (CN); Fang CHEN, ShenZhen (CN); Cuibin YAN, ShenZhen (CN); Jing JING, ShenZhen (CN); Peng MA, ShenZhen (CN); Xingtao JIANG, ShenZhen (CN); Xiaoling CHEN, ShenZhen (CN)

(21) Appl. No.: 18/926,845

(22) Filed: Oct. 25, 2024

(30) Foreign Application Priority Data
Nov. 2, 2023 (CN) 202311458266.6

(51) Int. Cl. A61K 9/00; A61K 31/216; A61K 31/4045; A61K 31/465; A61K 31/522; A61K 47/10; A61K 47/36; A61K 47/42

(52) U.S. Cl. CPC A61K 9/006; A61K 31/216; A61K 31/4045; A61K 31/465; A61K 31/522; A61K 47/10; A61K 47/36; A61K 47/42

(57) ABSTRACT

The present application provides a composition of oral soluble film and application thereof. The composition includes a film-forming agent, a plasticizing agent, and a target releasing substance. The film-forming agent includes pectin and polysaccharide backbones. The flexibility and mechanical strength of the oral soluble film may meet the production requirements during cutting and packaging processes, the adhesion force, the dissolving time limit, and the dissolving rate of the active components may also meet usage needs.

### FIELD

The subject matter relates to oral soluble films, and more particularly, to a composition of oral soluble film and application thereof.

### BACKGROUND

Oral soluble films, when being directly attached to different parts such as tongue, gingiva, and capsule, may be absorbed by the mucous membrane of mouth and quickly release into the blood system. The oral soluble films reduce metabolic loss of drugs and damages to the gastrointestinal tract, assist patients having difficulties swallowing, and eliminate the need of water to ingestion. The use of an oral soluble film is a good way for the delivery of drugs targeting nerves, blood vessels, pulmonary arteries, and even the heart, such as drugs for refreshing the mind, drugs or ingredients that have local effects in the oral cavity, and drugs or ingredients that need to avoid metabolism by gastrointestinal tract and hepatic portal.

However, most of the existing oral soluble films are cellulose films, which may be quickly disintegrated and dissolved within a release time duration which is less than 1 minute. Such films may also be difficult to dissolve and require a release time duration more than 2 hours, or even be insoluble. However, drugs or ingredients that are released too quickly may cause discomfort to the user. Also, some drugs or ingredients may require a sufficient dose for the recommended treatment. Some drugs or ingredients may need to stay in the oral cavity for 10 minutes, 30 minutes, or even longer time. The existing oral soluble films may not be applicable in real life situations.

Therefore, how to control the release or the residence time of the active components of the oral soluble film is problematic.

### SUMMARY

The present application provides a composition of oral soluble film, including a film-forming agent, a plasticizing agent, and a target releasing substance. The film-forming agent includes pectin and polysaccharide backbones.

The present application further provides an oral soluble film including the above composition.

The present application further provides a method for preparing an oral soluble film, including: mixing a film-forming agent, a plasticizing agent, and a target releasing substance with water to form a mixture, and stirring the mixture; subjecting the mixture to coating, heating and cutting to obtain the oral soluble film.

The present application further provides a drug or food including the above oral soluble film.

Other aspects and embodiments of the present disclosure are also expected. The above summary and the following detailed description are not intended to limit the present disclosure to any particular embodiment, but are merely intended to describe some embodiments of the present disclosure.

### BRIEF DESCRIPTION OF THE DRAWINGS

FIG. 1 is a flowchart of a method for preparing an oral soluble film according to an embodiment of the present application.

FIG. 2 is a flowchart of a method for preparing an oral soluble film according to another embodiment of the present application.

FIG. 3 illustrates a diagram of dissolving times and dissolving rates of oral soluble films prepared by examples 1 to 5 of the present application.

### DETAILED DESCRIPTION

The following descriptions are preferred embodiments of the present application. The embodiments are only for describing but not intended to limit the present application. It should be noted that for ordinary skilled in the art, several changes and modifications may be made within the principles of the embodiments of the present application. Such changes and modifications will still fall within the protection scope of the embodiments of the present application. For example, features described or illustrated in as part of one implementation can be used in another implementation to generate further implementation. It will, therefore, be appreciated that the embodiments described above may be modified within the scope of the claims.

In order to overcome at least one of the shortcomings mentioned above, a first aspect of the present application provides a composition, which includes a film-forming agent, a plasticizing agent, and a target releasing substance.

The film-forming agent includes pectin and polysaccharide backbone. In at least one embodiment, the film-forming agent includes a film-forming substrates such as starch and its derivatives, cellulose and its derivatives, chitosan, pullulan polysaccharide, and pectin.

The starch may be originated from corn, sweet potatoes, potatoes, cassava, rice, and wheat, and mainly includes amylose. The amylose is a starch formed by dehydration and condensation of two glucose molecules to form maltose and connecting the maltose by α-1,4-glycosidic bonds. The amylose has good film-forming properties and is tough, colorless, and tasteless.

The cellulose is another polysaccharide widely distributed in nature. The cellulose is a large molecular polysaccharide formed by connecting D-glucopyranose residues by β-1,4-glycosidic bonds, and also has good film-forming properties.

The chitosan, which has a chemical name of β-(1->4)-2-amino-2-deoxy-D-glucose, is formed by deacetylation of cell walls of fungi, algae, and chitin isolated from the shells of insects, crustaceans, and other animals. The chitosan has biodegradability and broad-spectrum antibacterial properties.

The sodium alginate is a linear polysaccharide extracted from brown algae. The sodium alginate is a linear block polymer compound without side chains (branches) formed by two monomers of β-D-mannuronic acid (M) and α-L-guluronic acid (G) connected by α-1,4-glycosidic bonds in three ways (MM segment, GG segment, and MG segment). The sodium alginate has good film-forming properties and water solubility.

The pullulan polysaccharide is a linear polymaltotriose formed by connecting glucose by two alpha-1,4 glycosidic bonds to form maltotriose and polymerizing the maltotriose by α-1,6 glycosidic bonds. The film prepared by the pullulan polysaccharide is transparent with a low oxygen transmission rate (OTR), and may inhibit the growth of fungi in the film to some extent.

The pectin is a polygalacturonic acid and mainly includes α-(1->4)-D-polygalacturonic acid that is partially methylated. The pectin also has good film-forming properties.

The plasticizing agent includes small molecules embedded into high polymers. The plasticizing agent may reduce the brittleness and hardness of the film, increase the distance between the high polymer molecules, enhance the flexibility of the film, and lower the glass transition temperature and increase the reaction speed of the high polymer molecules. In addition, the plasticizing agent may also affect other properties, such as mechanical properties and air permeability of the film.

In the present application, the starch and its derivatives, cellulose and its derivatives, chitosan, and pullulan polysaccharide are used as the polysaccharide backbone. The pectin and the polysaccharide backbone are functioned as the main film material. The pectin is filled in the polysaccharide backbone and cooperates with the plasticizing agent. By adjusting the ratio of the pectin to polysaccharide backbone, the polymerization degree and viscosity of the pectin may be adjusted, thereby adjusting the flexibility, adhesion, and adhesiveness of the polysaccharide backbone. The target releasing substance is wrapped by the pectin. After the pectin and the polysaccharide backbone form a film, the target releasing substance is dissolved under the function of a small amount of saliva. The more pectin, the higher the polymerization degree and the viscosity, the higher the film adhesion. Therefore, the dissolving time limit of the film material (that is, a time period required beaker for the film material to completely dissolve under a certain condition) increases, and the dissolving rate of the target releasing substance is slower. Therefore, the dissolving time limit of the film material in vitro and the dissolving time of the target releasing substance may both be adjusted, such that the oral soluble film may have a longer dissolving time limit.

When the composition of the present application is applied in an oral soluble film, the flexibility and mechanical strength of the oral soluble film may meet the production requirements during cutting and packaging processes, and the adhesion, adhesiveness, dissolving time limit, and dissolving properties of the active components also meet the actual usage needs.

In some embodiments, a mass percentage of the film-forming agent in the composition is in a range from 40% to 89.9% in order to meet the production and usage needs of the oral soluble film. In some embodiment, the mass percentage of the film-forming agent is in a range from 50% to 89.9%. In some embodiment, the mass percentage of the film-forming agent is in a range from 70% to 89.9%.

In some embodiments, the target releasing substance includes at least one of a water-soluble target releasing substance and an oil-soluble target releasing substance. In the present application, the film material formed by the pectin, the pullulan polysaccharide, and the plasticizing agent are both compatible with the water-soluble target releasing substance and the oil-soluble target releasing substance as active components. After the film material is mixed with the active components, the active components are soluble, and any crystalline precipitation of the active components is avoided.

In some embodiments, the target releasing substance may be active components that require delay of releasing time in the oral cavity, such as biologically active components for refreshing the mind. In some embodiments, the target releasing substance includes at least one of a water-soluble mind refreshing component and an oil-soluble mind refreshing component. The water-soluble mind refreshing component may be nicotine salt and chlorogenic acid. The oil-soluble mind refreshing component may be caffeine and melatonin.

In some embodiments, a mass percentage of the target releasing substance in the composition is in a range from 0.1% to 15%. In some embodiments, the mass percentage of the target releasing substance is in a range from 1% to 15%. In some embodiments, the mass percentage of the target releasing substance is in a range from 5% to 15%.

In some embodiments, the film-forming agent further includes at least one of xanthan gum and gelatin. The xanthan gum, also known as Hansheng gum, is a widely used microbial extracellular polysaccharide formed by fermentation of Xanthomnas campestris and carbohydrates (such as corn starch) as the main raw material. The xanthan gum has unique rheological properties, good water solubility, thermal stability, acid and base stability, and good compatibility with various salts.

The gelatin is a colorless to light yellow solid, and may be in form of powders, sheets, or blocks. The gelatin is glossy, odorless, and tasteless. The relative molecular weight of the gelatin is substantially in a range from 50000 to 100000, and the relative density of the gelatin is in a range from 1.3 to 1.4. The gelatin is insoluble in water, but may absorb 5 to 10 times of water to be expanded and soft when being immersed in water. The gelatin will transfer into a colloid when being heated, and will become gel when being cooled to a temperature of 35° C. to 40° C. When the water solution including the gelatin is boiled for a long time, the properties of the gelatin will change due to decomposition, and no gel will be formed after cooling. The gelatin is insoluble in ethanol, ether, and chloroform, and is soluble in hot water, glycerol, propylene glycol, acetic acid, salicylic acid, phthalic acid, urea, thiourea, thiocyanate, and potassium bromide.

In the present application, the xanthan gum or gelatin may be used to replace some pectin and filled in the polysaccharide backbone. The xanthan gum or gelatin may adjust the flexibility and mechanical strength of the polysaccharide backbone, and to adjust the dissolving time limit of the film material in vitro and the dissolving time of the target releasing substance.

In some embodiments, the plasticizing agent includes at least one of glycerol, propylene glycol, and polyethylene glycol. The plasticizing agent cooperates with the pectin, the xanthan gum, or the gelatin, thereby adjusting the flexibility and mechanical strength of the pullulan polysaccharide backbone and the dissolving time limit of the film material in vitro. In some embodiments, the polyethylene glycol may be at least one of polyethylene glycol 2000 and polyethylene glycol 4000.

In some embodiments, a mass percentage of the plasticizing agent in the composition is in a range from 10% to 20%. In some embodiments, the mass percentage of the plasticizing agent is in a range from 15% to 20%.

In some embodiments, the composition further includes at least one of an emulsifying agent, a flavoring agent, and a fragrance modifier. The emulsifying agent may enhance the emulsification of the target releasing substance or the fragrance modifier. The flavoring agent may adjust the overall flavor and taste, including sweet flavor and cool flavor. The fragrance modifier may adjust the fragrance, and may also adjust the mechanical properties (such as hardness) of the film material to allow the film material to meet the production needs.

In some embodiments, the emulsifying agent includes polysorbate (Tween), such as Tween 20, Tween 40, and Tween 80.

In some embodiments, a mass percentage of the emulsifying agent in the composition is in a range from 0% to 10%. In some embodiments, the mass percentage of the emulsifying agent is in a range from 5% to 10%. In some embodiments, the mass percentage of the emulsifying agent is in a range from 5% to 6%.

In some embodiments, the flavoring agent includes at least one of sucralose, N—[N-[3-(3-hydroxy-4-methoxyphenyl) propyl]-L-α-aspartyl]-L-phenylalanine 1-methyl ester (Edwin sweet), N—[N-(3,3-dimethylbutyl)-L-α-aspartyl]-L-phenylalanine 1-methyl ester (neotame), steviol glycoside, and N,2,3-trimethyl-2-isopropyl butamide (cooling agent WS-23).

In some embodiments, a mass percentage of the flavoring agent in the composition is in a range from 0% to 15%. In some embodiments, the mass percentage of the flavoring agent is in a range from 5% to 15%. In some embodiments, the mass percentage of the flavoring agent is in a range from 5% to 10%.

In some embodiments, the fragrance modifier includes at least one of Savarin essence, fruit modified essence, mint essence, watermelon essence, and strawberry essence.

In some embodiments, a mass percentage of fragrance modifier in the composition is in a range from 0% to 10%. In some embodiments, the mass percentage of fragrance modifier is in a range from 5% to 10%.

A second aspect of the present application provides an application of the above composition in the preparation of an oral soluble film.

A third aspect of the present application provides an application of the above composition in an oral soluble film.

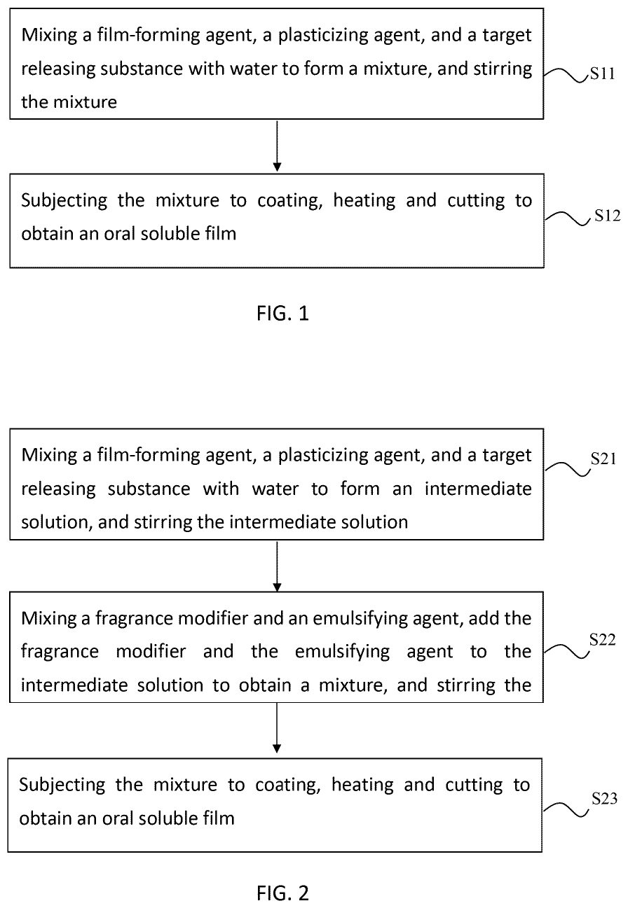

In some embodiments, when the composition includes a film-forming agent, a plasticizing agent, and a target releasing substance, a method for preparing the oral soluble film is provided as shown in FIG. 1. The method is provided by way of embodiments, as there are a variety of ways to carry out the method. Each block shown in FIG. 1 represents one or more processes, methods, or subroutines carried out in the method. Furthermore, the illustrated order of blocks may be changed. Additional blocks may be added or fewer blocks may be utilized, without departing from this disclosure. The method may begin at S11.

Step S11, the film-forming agent, the plasticizing agent, and the target releasing substance are mixed with water to form a mixture, and the mixture is stirred.

In some embodiment, the stirring speed is in a range from 300 r/min to 450 r/min, a stirring temperature is in a range from 60° C. to 80° C., and a stirring time duration is in a range from 30 min to 60 min.

Step S12, the mixture is subjected to coating, heating and cutting to obtain the oral soluble film.

In some embodiments, the heating is performed at a temperature in a range from 20° C. to 40° C. and a humidity in a range from 50% to 65%, for a time duration in a range from 40 min to 90 min.

In some embodiments, when the composition includes a film-forming agent, a plasticizing agent, a target releasing substance, and a fragrance modifier, a method for preparing an oral soluble film is shown in FIG. 2. The method is provided by way of embodiments, as there are a variety of ways to carry out the method. Each block shown in FIG. 2 represents one or more processes, methods, or subroutines carried out in the method. Furthermore, the illustrated order of blocks may be changed. Additional blocks may be added or fewer blocks may be utilized, without departing from this disclosure. The method may begin at S21.

Step S21, the film-forming agent, the plasticizing agent, and the target releasing substance are mixed with water to form an intermediate solution, and the intermediate solution is stirred.

The stirring speed of the intermediate solution is in a range from 300 r/min to 450 r/min, a stirring temperature is in a range from 60° C. to 80° C., and a stirring time duration is in a range from 30 minutes to 60 minutes.

Step S22, the fragrance modifier and the emulsifying agent are mixed and added to the intermediate solution to obtain a mixture. The mixture is stirred.

In some embodiments, the fragrance modifier and the emulsifying agent are mixed at a temperature in a range from 20° C. to 40° C.

In some embodiments, the stirring speed of the mixture is in a range from 300 r/min to 450 r/min, the stirring temperature is in a range from 50° C. to 80° C., and the stirring time duration is in a range from 30 minutes to 60 minutes.

Step S23, the mixture is subjected to coating, heating and cutting to obtain the oral soluble film.

In some embodiments, before the coating of the mixture, the mixture undergoes a defoaming treatment to avoid bubbles on the film after the coating.

A fourth aspect of the present application provides an oral soluble film, which includes the composition mentioned above and moisture.

A mass percentage of the moisture in the oral soluble film is in a range from 7% to 13%. A tensile strength of the oral soluble film is in a range from 6 N/mm² to 20 N/mm². A breaking elongation percentage of the oral soluble film is larger than 30%. An adhesion force of the oral soluble film is in a range from 1 N/cm² to 3 N/cm². A content of the target releasing substance in the oral soluble film is in a range from 1% to 10%. The dissolving time limit of the oral soluble film in vitro is in a range from 20 minutes to 45 minutes. The dissolving time for the oral soluble film to release 80% of the target releasing substance is 5 minutes to 10 minutes.

The flexibility and mechanical strength of the oral soluble film in the present application may meet the production requirements during the cutting and packaging processes, and the adhesion, adhesiveness, dissolving time limit, and dissolving properties of the active components also meet the actual usage needs.

A fifth aspect of the present application provides a drug or food including the oral soluble film mentioned above, which may prolong the dissolving time of the target releasing substance.

The following will provide a detailed description of the implementations of the present application by different examples. The reagents and the acquisition methods involved in the following examples of the present application are shown in Table 1.

TABLE 1

| | |
|---|---|
| Raw material- Savarin concentrated essence | Savarin ™ 100HY |
| Raw material-chemical-fruit modified essence | Guangzhou Leda LC230532 |
| Raw material-chemical- sweetener (steviol glycoside) | Laiyin 97% SRE10, 97% |
| Raw material- chemical- sweetener (Edwin sweet) | Wuhan Huasweet Biotechnology Co., Ltd |
| Raw material- chemical- pectin | Chongqing Mengtai Biotechnology Co., |
| | Ltd MTL500 |
| Raw material- chemical- pullulan polysaccharide | Shangdong Freda Biotechnology Co., Ltd |
| Raw material- chemical-PEG 400 | AOKI Co., Ltd |
| Raw material- chemical- Tween 20 | Sunvo Biotechnology F1734FL311 |

The oral soluble film, the method for preparing the same, the test method, and the method used in the example are as follows:

### 1. Composition:

a film-forming agent of 40% to 89.9%, including pullulan polysaccharide and pectin;
a plasticizing agent of 10% to 20%, such as glycerol and polyethylene glycol 400;
an emulsifying agent of 0% to 10%, such as Tween 20, which was used to improve the emulsification degree of the essence or the active substance;
a sweetener or cooling agent of 0% to 15%, such as Edwin sweet, neotame, steviol glycoside, and cooling agent WS-23, which was used to adjust the taste and flavor;
an essence of 0% to 10%, such as Savarin essence, fruit modified essence, and some specific plant or fruit essence (e.g., mint essence, watermelon essence, and strawberry essence), which was used to adjust the taste and sense of smell;
a target releasing substance of 0.1% to 15%, which includes ingredients for refreshing the mind, such as at least one of nicotine salt, caffeine, melatonin, and chlorogenic acid.

### 2. Preparation Method:

(1) Mixing and stirring step: the powders of pullulan polysaccharide, pectin, sweetener, plasticizing agent, and active components were weighed and mixed, and an appropriate amount of water, usually 4 to 5 times of the weight of the powders, were added to the powders; a target temperature of a water bath was set to be 60° C. to 80° C., and the material was stirred at 300 to 450 r/min for 30 to 60 minutes until there were no obvious particles or agglomerations; the essence and the emulsifying agent were mixed at room temperature for 10 min and then added to the material at 300 to 450 r/min, and the temperature of the water bath may be reduced to 50° C. to 60° C. if necessary; the material was stirred for 30 to 60 min, and after the stirring, the material was taken out and remained at room temperature for defoaming.
(2) Casting coating step: after that the entire material had cooled to about 25° C. and there were no fine bubbles by observation, a measuring cup or other tool was used to take and pour the material onto an appropriate position of a coating table; a reasonable coating height was set to be about 800 μm, and the coating speed was set to be 1 mm/min for coating.
(3) heating and cutting process: after the coating, the film was heated at an ambient temperature of 25° C. and a humidity of 50% to 65%, and the heating temperature was 60° C. to 65° C., and the heating time duration was 40 to 90 minutes; the heating was finished based on the properties of the film; then, a food-grade cutting knife or grinding tool was used to cut the film to an appropriate size of 2 cm× (1-3) cm, and the product was subjected to package and storage.

### 3. Test and Evaluation Methods for the Oral Soluble Film:

After the tests and sensory evaluation on commercial product and the products of the present application, the oral soluble film is determined to be suitable for cutting and packaging processes and able to provide a good attachment sensation. The physical and chemical properties of the oral soluble film are shown in Table 2.

TABLE 2

| Items | Technical standard |
|---|---|
| Mass percentage of moisture | 7%-13% |
| Tensile strength | 6 N/mm²-20 N/mm² |
| Breaking elongation percentage | >30% |
| Adhesion force | 1 N/cm²-3 N/cm² |
| Mass percentage of API (single piece) | 1%-10% |
| Dissolving time limit in vitro | 20 min to 45 min |
| Dissolving time of 80% API | 5 min-10 min |

### 4. Test Method:

Moisture: the mass percentage of the entire oral soluble film was tested according to the Karl Fischer method;

Thickness: a sample of the oral soluble film was taken, and the outer packaging was removed; points in the middle and four edges of the oral soluble film were selected, and the thickness at each point was measured by a micrometer, and the average thickness value was calculated (five-point-method).

Weight: a sample of the oral soluble film was taken, and the outer packaging was removed; a tweezer was used to clamp the entire oral soluble film, and the oral soluble film was placed on an electronic balance to record the weight; the weights of 10 samples were measured as mentioned above, and the mean and variance values were calculated accordingly.

Tensile strength: the oral soluble film was clamped by two clamps with rubber or cotton pads to avoid cutting of the oral soluble film, and the oral soluble film was fixed on the clamping position of the TA-XT2 texture analyzer; a distance between the two clamps was set to be less than the length of the oral soluble film; the upper clamp was moved upward at a speed of 20-120 mm·min⁻¹ until the oral soluble film breaks; the tensile strength TS (MPa), i.e., a maximum stress applied onto the film, was calculated by the formula: TS=Fₘₐₓ/A, A=t×s, wherein Fₘₐₓ denotes the maximum force when the oral soluble film breaks (N), A denotes the initial cross-sectional area of the oral soluble film (mm²), t denotes the thickness of the oral soluble film, and s denotes the width of the oral soluble film.

Breaking elongation percentage: the oral soluble film was clamped by two clamps with rubber or cotton pads to avoid cutting of the oral soluble film, and the oral soluble film was fixed on the clamping position of the TA-XT2 texture analyzer; a distance between the two clamps was set to be less than the length of the oral soluble film; the upper clamp was moved upward at a speed of 20-120 mm min⁻¹ until the oral soluble film breaks; the breaking elongation percentage (L %) was by the formula: L %=100×(Lₘₐₓ/L−1), wherein Lₘₐₓ is the distance between the two clamps when the oral soluble film breaks (mm), and L is the initial distance between the two clamps (mm).

Adhesion force in vitro: two rubbers were fixed on the end portion and the base of the tension meter, respectively; a sample of the oral soluble film was taken, and the outer packaging was removed; the length L and width W of the oral soluble film were measured; artificial saliva of 50 μl was taken to moisten the surface of the rubber; the oral soluble film was attached to the surface of the rubber, ensuring that the length and width of the rubber were greater than the length and width of the oral soluble film, respectively; the oral soluble film was flattened, and the surface of another rubber moistened with artificial saliva of 50 μl was placed onto the oral soluble film; an object of 2 kg was used to press the oral soluble film with the two rubbers for 90 s, and then the object was removed; the wheel was rotated to separate the two rubbers within 5 s, and the peak value displayed by the tension meter was the maximum adhesion force of the oral soluble film (Fₘₐₓ); the adhesion force was calculated by the formula: F=Fₘₐₓ/S, S=L×W, wherein Fₘₐₓ denotes the maximum force of the oral soluble film (N), S denotes the initial area of the oral soluble film (cm²), L denotes the length of the oral soluble film, and W denotes the width of the oral soluble film.

Dissolving time limit: artificial saliva of 100 mL was placed into a beaker, and the beaker was placed on an electromagnetic stirrer; the beaker was heated by a 37° C. constant temperature water bath with a stirring speed of 130 r/min; an oral soluble film was flattened on a grid, the grid was plated on a bracket and hanged on the artificial saliva, ensuring that the artificial saliva contacts but does not fully infiltrate the oral soluble film; the dissolving time of the oral soluble film (T) was recorded.

Test of active components: an oral soluble film was weighed and completely dissolved in artificial saliva of 100 mL; an appropriate reagent kit, liquid chromatography, and other method was used to measure the active components; the content of the active components was calculated by the formula: 100×content of components×100 mL.

Dissolving curve: an oral soluble film was placed on a mesh carrier and suspended on the beaker containing artificial saliva of 100 mL, ensuring that the oral soluble film was infiltrated by the artificial saliva; the beaker was placed on a temperature-controlled magnetic stirrer, the stirring speed was set to be 130 r/min and the temperature was set to be 37° C.; after specified time durations such as 0.5 min, 1 min, 2 min, 3 min, 5 min, 8 min, 12 min, 18 min, 25 min, and 35 min, the sample of 100 μL was collected and the content of the active components therein was measured; the dissolving rate of the active components was calculated, and a curve of the dissolving characteristics of the active components was drawn based on the content of the active components.

### Example 1

Composition and water were prepared according to Table 3, and the oral soluble film was prepared according to the above method. The mass ratio of the composition to the water was 23.7:76.3.

TABLE 3

| Ingredients | Mass percentage (%) |
|---|---|
| pullulan polysaccharide | 11.84/23.7 |
| pectin | 6.57/23.7 |
| polyethylene glycol | 3.82/23.7 |
| nicotine salt diluent | 1.47/23.7 |

### Example 2

Composition and water were prepared according to Table 4, and the oral soluble film was prepared according to the above method. The mass ratio of the composition to the water was 21.6:78.4.

TABLE 4

| Ingredients | Mass percentage (%) |
|---|---|
| pullulan polysaccharide | 11.66/21.6 |
| pectin | 3.38/21.6 |
| glycerol | 4.76/21.6 |
| melatonin | 1.8/21.6 |

### Example 3

Composition and water were prepared according to Table 5, and the oral soluble film was prepared according to the above method. The mass ratio of the composition to the water was 24.2:75.8.

TABLE 5

| Ingredients | Mass percentage (%) |
|---|---|
| pullulan polysaccharide | 11.8/24.2 |
| pectin | 3.42/24.2 |
| polyethylene glycol | 3.8/24.2 |
| nicotine salt diluent | 1.52/24.2 |
| sweet and cool flavoring agent | 1.4/24.2 |
| mint essence | 1.3/24.2 |
| Tween | 1.0/24.2 |

### Example 4

Composition and water were prepared according to Table 6, and the oral soluble film was prepared according to the above method. The mass ratio of the composition to the water was 23.57:76.43.

TABLE 6

| Ingredients | Mass percentage (%) |
|---|---|
| pullulan polysaccharide | 11.84/23.57 |
| pectin | 3.44/23.57 |
| polyethylene glycol | 4.82/23.57 |
| nicotine salt diluent | 1.47/23.57 |
| sweeter | 1.2/23.57 |
| strawberry essence | 0.8/23.57 |
| Tween | 1.0/23.57 |

### Example 5

Composition and water were prepared according to Table 7, and the oral soluble film was prepared according to the above method. The mass ratio of the composition to the water was 23.65:76.35.

TABLE 7

| Ingredients | Mass percentage (%) |
|---|---|
| pullulan polysaccharide | 11.84/23.65 |
| pectin | 1.44/23.65 |
| polyethylene glycol | 2.58/23.65 |
| glycerol | 2.82/23.65 |
| nicotine salt diluent | 2.47/23.65 |
| sweeter | 1.2/23.65 |
| watermelon essence | 1.3/23.65 |
| Tween | 2.0/23.65 |

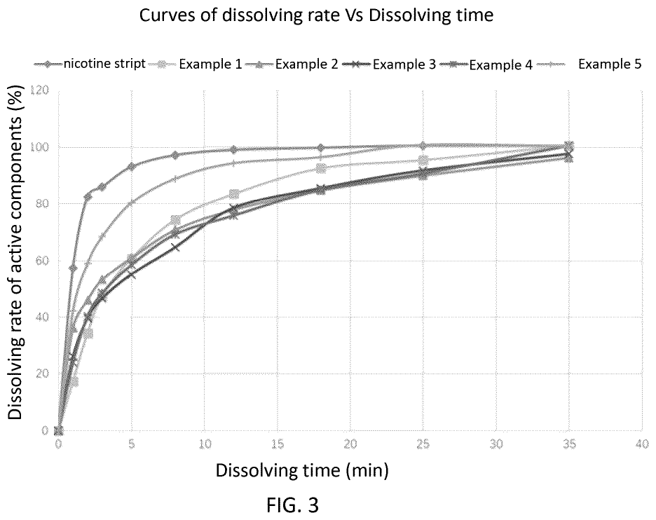

The test results of the performances of the oral soluble film prepared by examples 1-5 are shown in FIG. 3 and Table 8.

TABLE 8

| | | | Breaking | | Mass | 100% |
|---|---|---|---|---|---|---|
| | | Tensile | elongation | Adhesion | percentage | dissolving |
| | Thickness | strength | percentage | force | of moisture | time limit in |
| Number | (μm) | (N/mm²) | (%) | (N/cm²) | (%) | vitro (min) |
| Example 1 | 113.3 | 19.91 | 33.3 | 1.6 | 9.5 | 35 |
| Example 2 | 121.33 | 12.65 | 53.67 | 0.94 | 11.04 | 40 |
| Example 3 | 123.33 | 11.27 | 64.27 | 1.19 | 11.52 | 40 |
| Example 4 | 116.67 | 12.38 | 68.17 | 1.01 | 10.37 | 35 |
| Example 5 | 108.52 | 14.32 | 127.6 | 1.65 | 11.03 | 20 |

The dissolving characteristics of the active components of the oral soluble film prepared by examples 1-5 are shown in Table 9.

TABLE 9

| | Dissolving rate (%) | | | | | |
|---|---|---|---|---|---|---|
| | Commercial | | | | | |
| Duration | oral soluble | Example | Example | Example | Example | Example |
| (min) | film | 1 | 2 | 3 | 4 | 5 |
| 0 | 0 | 0 | 0 | 0 | 0 | 0 |
| 1 | 57.34 | 17.346 | 36.13 | 25.92 | 23.82 | 42.126 |
| 2 | 82.47 | 34.424 | 45.93 | 39.61 | 40.22 | 58.928 |
| 3 | 85.85 | 47.528 | 53.14 | 46.52 | 48.41 | 68.35 |
| 5 | 92.93 | 60.7 | 60.74 | 55.02 | 58.40 | 80.267 |
| 8 | 97.06 | 74.34 | 70.71 | 64.53 | 69.19 | 88.847 |
| 12 | 98.86 | 83.471 | 77.77 | 78.40 | 77.82 | 94.307 |
| 18 | 99.63 | 92.53 | 84.72 | 85.40 | 85.90 | 96.41 |
| 25 | 100.42 | 95.43 | 89.90 | 91.63 | 90.70 | 100.714 |

In summary, when the present application uses the composition to prepare an oral soluble film, the flexibility and mechanical strength of the oral soluble film may meet the production requirements during cutting and packaging processes, the adhesion force is improved, and the dissolving time limit is increased, which has a lower dissolving rate of the active components compared to the commercial oral soluble film. Thus, the present application may meet the requirements of the release time of the active components in the oral cavity for refreshing the mind.

The above embodiments are only for describing but not intended to limit the present disclosure. Although the embodiments of the present disclosure have been described, those having ordinary skill in the art can understand that changes may be made within the principles of the present disclosure, up to and including the full extent established by the broad general meaning of the terms used in the claims. It will, therefore, be appreciated that the embodiments described above may be modified within the scope of the claims.

What is claimed is:

1. A composition of oral soluble film comprising:
a film-forming agent;
a plasticizing agent; and
a target releasing substance,
wherein the film-forming agent comprises pectin and polysaccharide backbone.

2. The composition according to claim 1, wherein the film-forming agent further comprises at least one of xanthan gum and gelatin.

3. The composition according to claim 1, wherein the plasticizing agent comprises at least one of glycerol and polyethylene glycol.

4. The composition according to claim 1, wherein the composition satisfies at least one of following features (1) and (2):
(1) the target releasing substance comprises at least one of at least one of a water-soluble target releasing substance and an oil-soluble target releasing substance;
(2) the target releasing substance comprises at least one of nicotine salt, caffeine, melatonin, and chlorogenic acid.

5. The composition according to claim 1, wherein the polysaccharide backbone comprises at least one of starch and its derivatives, cellulose and its derivatives, chitosan, pullulan polysaccharide, and pectin.

6. The composition according to claim 1, further comprising at least one of an emulsifying agent, a flavoring agent, and a fragrance modifier.

7. The composition according to claim 6, wherein the composition satisfies at least one of following features (1) to (3):
(1) the emulsifying agent comprises polysorbate;
(2) the flavoring agent comprises at least one of sucralose, steviol glycoside, N—[N-[3-(3-hydroxy-4-methoxyphenyl) propyl]-L-α-aspartyl]-L-phenylalanine 1-methyl ester, N—[N-(3,3-dimethylbutyl)-L-α-aspartyl]-L-phenylalanine 1-methyl ester, and N,2,3-trimethyl-2-isopropyl butamide;
(3) the fragrance modifier comprises at least one of Savarin essence, fruit modified essence, mint essence, watermelon essence, and strawberry essence.

8. The composition according to claim 1, wherein the composition satisfies at least one of following features (1) to (3):
(1) a mass percentage of the film-forming agent in the composition is in a range from 40% to 89.9%;
(2) a mass percentage of the plasticizing agent in the composition is in a range from 10% to 20%;
(3) a mass percentage of the target releasing substance in the composition is in a range from 0.1% to 15%.

9. The composition according to claim 6, wherein the composition satisfies at least one of following features (1) to (3):
(1) a mass percentage of the emulsifying agent in the composition is in a range from 0% to 10%;
(2) a mass percentage of the flavoring agent in the composition is in a range from 0% to 15%;
(3) a mass percentage of fragrance modifier in the composition is in a range from 0% to 10%.

10. An oral soluble film comprising:
a composition comprising a film-forming agent, a plasticizing agent, and a target releasing substance,
wherein the film-forming agent comprises pectin and polysaccharide backbones.

11. The oral soluble film according to claim 10, wherein the film-forming agent further comprises at least one of xanthan gum and gelatin.

12. The oral soluble film according to claim 10, wherein the plasticizing agent comprises at least one of glycerol and polyethylene glycol.

13. The oral soluble film according to claim 11, wherein the composition satisfies at least one of following features (1) and (2):
(1) the target releasing substance comprises at least one of at least one of a water-soluble target releasing substance and an oil-soluble target releasing substance;
(2) the target releasing substance comprises at least one of nicotine salt, caffeine, melatonin, and chlorogenic acid.

14. The oral soluble film according to claim 10, wherein the polysaccharide backbone comprises at least one of starch and its derivatives, cellulose and its derivatives, chitosan, pullulan polysaccharide, and pectin.

15. The oral soluble film according to claim 10, wherein the oral soluble film satisfies at least one of following features (1) to (7):
(1) the oral soluble film further comprises moisture, and a mass percentage of the moisture in the oral soluble film is in a range from 7% to 13%;
(2) a tensile strength of the oral soluble film is in a range from 6 N/mm² to 20 N/mm²;
(3) a breaking elongation percentage of the oral soluble film is larger than 30%;
(4) an adhesion force of the oral soluble film is in a range from 1 N/cm² to 3 N/cm²;
(5) a content of the target releasing substance in the oral soluble film is in a range from 1% to 10%;
(6) a dissolving time limit of the oral soluble film in vitro is in a range from 20 minutes to 45 minutes;
(7) a dissolving time for the oral soluble film to release 80% of the target releasing substance is 5 minutes to 10 minutes.

16. A method for preparing an oral soluble film, the method comprising:
mixing a film-forming agent, a plasticizing agent, and a target releasing substance with water to form a mixture, and stirring the mixture, wherein the film-forming agent comprises pectin and polysaccharide backbones; and
subjecting the mixture to coating, heating and cutting to obtain the oral soluble film.

17. The method according to claim 16, wherein mixing the film-forming agent, the plasticizing agent, and the target releasing substance with water to form the mixture comprises:
mixing the film-forming agent, the plasticizing agent, and the target releasing substance with water to form an intermediate solution;
mixing a flavoring agent and an emulsifying agent, adding the flavoring agent and the emulsifying agent into the intermediate solution, and stirring the intermediate solution to obtain the mixture.

18. The method according to claim 17, wherein the method further comprises at least one of following features (1) to (3):
(1) for the intermediate solution, a stirring speed is in a range from 300 to 450 r/min, a stirring temperature is in a range from 60° C. to 80° C., and a stirring time duration is in a range from 30 minutes to 60 minutes;
(2) for the mixture, a stirring speed is in a range from 300 r/min to 450 r/min, a stirring temperature is in a range from 50° C. to 80° C., and a stirring time duration is in a range from 30 minutes to 60 minutes;
(3) the flavoring agent and the emulsifying agent are mixed at a temperature in a range from 20° C. to 40° C.

19. The method according to claim 16, wherein the method further comprises at least one of following features (1) and (2):
(1) before subjecting the mixture to coating, heating and cutting, the method further subjecting the mixture to defoaming treatment;
(2) the heating is performed at a temperature of 20° C. to 40° C. and a humidity of 50% to 65% for a time duration of 40 minutes to 90 minutes.

20. A drug or food, comprising the oral soluble film according to claim 10.
